(19) United States
(12) Patent Application Publication
(10) Pub. No.: US 2004/0258776 A1
(43) Pub. Date: Dec. 23, 2004

(54) Composition comprising aleurone, method of administering the same, and method of manufacturing the same

(75) Inventors: Arturo Bohm, Oberuzwil (CH); Rudolf Buri, Reinach (CH)

Correspondence Address: GREENBLUM & BERNSTEIN, P.L.C., 1950 ROLAND CLARKE PLACE, RESTON, VA, 20191, US

(73) Assignee: Buhler AG, Uzwil (CH)

(21) Appl. No.: 10/853,149

(22) Filed: May 26, 2004

Related U.S. Application Data
(63) Continuation-in-part of application No. PCT/CH02/00567, filed on Oct. 17, 2002.

(30) Foreign Application Priority Data
Nov. 26, 2001 (DE) 101 57 626.9

(51) Int. Cl. A01H 1/00; A61K 35/78

(52) U.S. Cl. 424/750; 800/320

(57) ABSTRACT

This invention is directed to a composition comprising at least one aleurone component and aleurone, extracted from bran and effective substances contained in the aleurone and their favourable physiological properties are supplied to a human or animal in an easily accessible form, as well as a method of manufacturing thereof. This composition can be administered to a human or animinal. The composition of the present invention can be manufactured by adding the aleurone component and/or the aleurone to a food component or fodder component in a liquid or powder form which is easily accessible to the human or animal organism.

### CROSS-REFERENCE TO RELATED APPLICATIONS

The present application is a Continuation-in-Part of International Patent Application No. PCT/CH02/00567 filed Oct. 17, 2002, and claims priority of German Patent Application No. 101 57 626.9 filed Nov. 26, 2001. Moreover, the disclosure of the International Patent Application No. PCT/CH02/00567 is expressly incorporated by reference herein in its entirety.

### BACKGROUND OF THE INVENTION

1. Field of the Invention

The invention relates to means of supplying the body system with aleurone.

2. Discussion of Background Information

From nutritional/physiological aspects, aleurone is an extremely valuable component of grain, and to a particular extent of wheat, and is located in the wheat in a single-cell layer (aleurone layer) between the flour body (endosperm) and the shell (pericarp and testa) of the wheat. The average weight proportion of aleurone in the wheat is 8.3%.

Aleurone is isolated from bran by means of physical or, in particular, mechanical/abrasive and biological/enzymatic processes, and subjected to further treatment. Reference is made at this stage to international patent application PCT/CH 01/00506 to Bohm et al. in the name of the same Assignee, which is expressly incorporated by reference herein in its entirety.

In the aleurone cells of the wheat, all nutritionally/physiologically significant substances of wheat are contained in concentrated form, for example, vitamins, mineral substances, essential fatty acids, food fibers, high-quality protein (albumin), and special protective substances (bioactive substances, such as phenoles, lignan, phytin, etc.). Moreover, wheat has been used for several thousand years as a tried-and-tested food for both animals and human beings.

The high content of vitamins and mineral substances contribute to a healthy state in general and to mental and physical productivity.

The food fibers of the aleurone help to improve digestive activities. In particular, they slow down resorption, and thus promote a lasting feeling of repletion.

Aleurone is easily digestible. A specific point is that it contains no sticky proteins (gliadin, glutenin), and is thus also suitable for those suffering from wheat allergy or coeliac disease.

Bioactive substances of aleurone, such as polyphenols, flavanoids, lignan, beta-glucan etc., have protective effects against a number of illnesses caused by civilization, such as the hardening of the arteries and cancer.

The object of the invention is to enable the use of aleurone and its components in human and animal nutrition.

The object of the invention is accomplished by a means of supplying aleurone components and/or aleurone, extracted from bran, in particular wheat bran, when the effective substances contained in the aleurone and thus their favourable physiological properties are supplied to the human or animal organism in an easily accessible form. As a result, the nutritional/physiological components of the aleurone can be better absorbed by the organism than upon the consumption of whole grain.

Other exemplary embodiments and advantages of the present invention may be ascertained by reviewing the present disclosure.

### SUMMARY OF THE INVENTION

The present invention is directed to a composition comprising at least one aleurone component and aleurone extracted from bran, and effective substances contained in the aleurone and their favourable physiological properties are supplied to a human or animal in an easily accessible form. The bran of the composition can be a wheat bran, and the aleurone component can be contained in relative proportion with the wheat bran. The aleurone component of the present invention can be different aleurone components contained in relative proportions which correspond to or deviate from their natural relative proportions in the bran.

The aleurone component can be in the form of aleurone cell groups. The aleurone cell group can comprise in an average up to about 100 aleurone cells, preferably up to about 5 to about 50 aleurone cells, and more preferably up to about 10 to about 20 aleurone cells. The aleurone cells can have weakened cell walls. The walls of the aleurone cells can be weakened through at least one of enzymatic influence, mechanical influence, chemical influence or thermal influence, wherein the thermal influence is a steam treatment and/or microwave treatment.

The aleurone components of the present invention can be in the form of destroyed aleurone cells, and comprise both the total cell content and the entire destroyed cell walls of the aleurone. The aleurone component of the present invention can be in the form of destroyed aleurone cell, and comprises at least one part of the entire cell content and/or at least one part of the destroyed cell wall of the aleurone.

The composition of the present invention may contain no other components, and wherein the aleurone component and/or aleurone can comprise aleurone cell groups is in flake, granule, pressling or powder form. The aleurone component and/or aleurone can contain at least one destroyed aleurone cell. The aleurone component and/or aleurone can comprise only cell content which originates from aleurone cells in liquid form. The cell content can be thickened to make a thick fluid. The cell content can be diluted to make a runny fluid.

The aleurone component and/or aleurone can comprise only the cell content or only the destroyed cell wall which originates from aleurone cell group in its dried form. The dried form can be in flake, granule, pressling or powder form. The aleurone component and/or aleurone can comprise only destroyed cell walls originating from aleurone cells and water.

The composition of the present invention can further comprise a food component. The food component can be at least one of:

a) basic food which is at least one of flour or semolina made from wheat, rye, rice, corn or soya; fermented or non-fermented milk; animal or vegetable fats; fruit or fruit juice; vegetable or vegetable juice; pure water, mineral water or other enriched water; meat; and fish; and

b) processed food which is at least one of cheese, quark or yoghurt; beverages, milk, or juices; bakery products, bread or pastry; noodles or pasta; confectionery, chocolate, or jelly; breakfast cereals or bars; sandwich spreads; meat substitute; and sausage; and special food with specific effects which is at least one of functional food; pharma food; designer food; and nutraceuticals.

The fodder component can be at least one of (a) food for domestic animals or pets which is at least one of dog food; cat food; rodent food; fish food; reptile food; amphibian food; bird food; and (b) fodder for livestock which is at least one of horse fodder; cattle fodder; pig fodder; sheep fodder; and poultry fodder.

The composition of the present invention can further comprise a carrier which is neutral to the metabolism. The carrier can be carrier (1) an indigestible carrier; or (2) a tasty carrier, wherein the ingestible carrier comprises at least one agent which is at least one of insoluble salts, waxes, starch, gelatine, and chewing-gum; and wherein the tasty carrier comprises at least one agent which is at least one of dextrose, maltose, and fruit gum.

The composition of the present invention can be manufactured by adding the aleurone component and/or the aleurone to a food component or fodder component in a liquid or powder form which is easily accessible to the human or animal organism. The composition of the present can also be manufactured by adding at least one aleurone component and aleurone to a food component or fodder component in a liquid or powder form which is easily accessible to the human or animal organism, wherein after the addition of the aleurone component to the food component, the food is subjected to treatment to weakening or further weakening of the aleurone component.

The composition of the present can also be manufactured by adding the aleurone component and/or the aleurone to a processed food or processed fodder during its processing, in a liquid or powder form which is easily accessible to the human or animal organism. The composition of the present invention can also be manufactured by adding at least one weakened aleurone cells and unweakened aleurone cells to a food product, and subjecting the food product to at least one type of weakening treatment to weaken or further weaken the aleurone cells.

Alternatively, the composition of the present invention can be manufactured by adding intact aleurone or intact aleurone cells or aleurone cell groups to a processed food or a processed fodder during a procedural step of processing, when the food or fodder is exposed to at least one of enzymatic influence, mechanical influence, thermal influence, and chemical influence, wherein the mechanical influence is shearing, compression or expansion, and wherein the thermal influence is steam and/or microwave.

The composition of the present invention can be manufactured by adding the entire quantity of the aleurone component and/or the aleurone to the food component or fodder component in more than one step.

The composition of the present invention can also be manufactured by constantly adding to the food component or the fodder component in at least one of the following forms during the processing: a) aleurone particle powder comprising aleurone cells and/or aleurone cell groups; b) aleurone juice comprising aleurone cell plasma; c) suspended aleurone cells and/or aleurone cell groups; d) diluted aleurone cell plasma; e) thickened aleurone cell plasma; f) granules, pellets, or powder comprising dried aleurone cell plasma.

The present invention is also directed to administering to a human or animinal the composition of the present invention. The composition of the present invention can be administered to a human or animal organism wherein the composition is a food supplement fodder supplement. The present invention can also include a method of administering to a human or animal a composition manufactured by adding at least one weakened aleurone cells and unweakened cells to a food product, and subjecting the food product to at least one type of weakening treatment to weaken or further weaken the aleurone cells.

The food component can be a process food component, and wherein the fodder component is a process fodder component. The food component or fodder component can be exposed to at least one of enzymatic influence, mechanical influence, thermal influence, and chemical influence.

The entire quantity of the aleurone component and/or the aleurone can be added to the food component or fodder component in more than one step. The food component can be a food supplement, and wherein the fodder supplement is a fodder supplement.

### BRIEF DESCRIPTION OF THE DRAWINGS

The present invention is further described in the detailed description which follows, in reference to the noted plurality of drawings by way of non-limiting examples of exemplary embodiments of the present invention, in which like reference numerals represent similar parts throughout the several views of the drawings, and wherein:

FIG. 1 shows a microscopic view through the aleurone layer which separates the endosperm from the hull of the grain.

FIG. 2 shows aleurone cell clusters (ASP-1) suspended in water together with starch and hull fragments.

FIG. 3 shows damaged aleurone cells (ASP-2) suspended in water in the form as aleurone cell fragments.

FIG. 4 shows the particle size distribution of the aleurone type shown in FIG. 2 and of the aleurone type shown in FIG. 3.

### DETAILED DESCRIPTION OF THE PRESENT INVENTION

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show structural details of the present invention, the description making apparent to those skilled in the art how the several forms of the present invention may be embodied in practice.

In the form concerned, the various aleurone components are preferably contained in relative proportions which correspond to their natural relative proportions in bran, particularly wheat bran. In view of the fact that, despite a severe re-construction of our environment and way of life, the genetic make-up of a human being and numerous animals and livestock has changed to only a slight extent, it can be assumed that this “natural mix relationship” is for the organism and its physiological reactions still fairly well “geared to needs”.

The various aleurone components can, as required, be contained in relative proportions which deviate from their natural relative proportions in bran, in particular wheat bran.

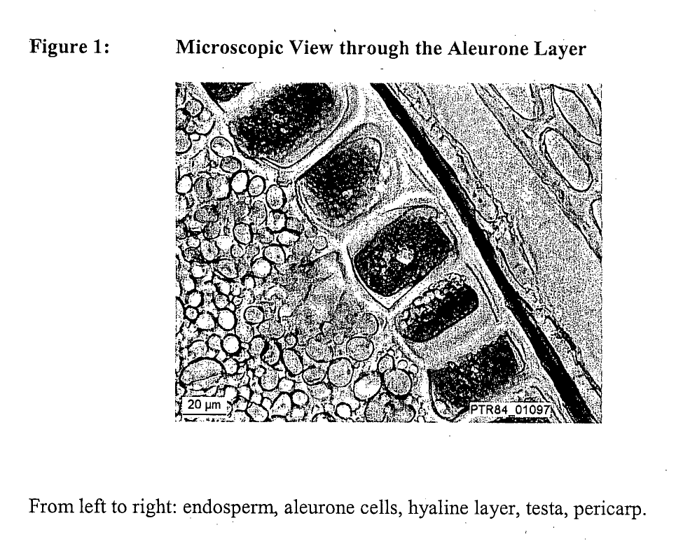

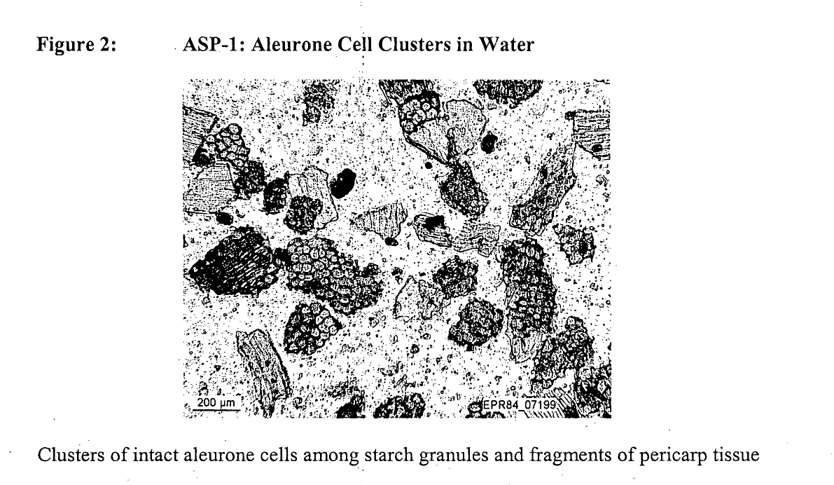

Moreover, the aleurone components can be in the form of aleurone cell groups/clusters, which comprise up to 100 aleurone cells, in particular around 5 to 50 aleurone cells on average, and preferably around 10 to 20 aleurone cells. This form of aleurone can, on the one hand, be extracted economically from the bran, and easily and evenly distributed in foods, on the other. Clusters can include groups of cells that are adjacent together. Those clusters are fragments of the aleurone layer, such as those are shown in FIGS. 1 and 2, with the intact layer being shown in FIG. 1, and clusters being shown in FIG. 2.

Appropriately, some of the aleurone cells of the aleurone cell groups can have weakened cell walls. A weakening of the cell walls which are difficult to digest supports digestion and facilitates the organism's access to the valuable aleurone components. A particular point is that the walls of the aleurone cells can have been weakened by enzymatic, mechanical or chemical means, or e.g., thermal means through steam treatment and/or microwave treatment. Combinations of these types of cell wall weakening are also advantageous.

Where required, the aleurone components can be contained in the form of destroyed aleurone cells (“aleurone pulp”) and comprise both the total cell content and the total destroyed cell walls of the aleurone content. In this form, the aleurone is practically “pre-digested”, and the components of the aleurone cell content can be absorbed particularly easily by the organism, without the organism being deprived of the physiological benefit of the destroyed cell walls of the aleurone. Here again, the aleurone components can, if required, be contained in the form of destroyed aleurone cells (“cell mush”), and comprise at least a part of the total cell content and/or at least a part of the disturbed cell walls of the aleurone content.

A particular means of supplying the aleurone components and/or the aleurone according to the invention does not comprise any further components apart from aleurone components, and thus merely consists of aleurone. This form of administration is suitable as a food supplement or fodder supplement. It can comprise aleurone cell groups, particularly in flake, granule, pressling or powder form, and can, where required, contain at least partly destroyed aleurone cells (“aleurone pulp”).

The aleurone components and/or the aleurone in this form of administration can also comprise the cell content originating from the aleurone cells in its liquid form (“aleurone juice”), which, preferably, has been thickened to make a syrupy fluid (“aleurone syrup”), or diluted to make a runny fluid (“aleurone water”).

Appropriately, they consist only of the cell content originating from aleurone cells in its dried form, in particular, in flake, granule, pressling or powder form.

Analogously, they can also comprise only the destroyed cell walls originating from aleurone cells, in particular in flake, granule, pressling or powder form, when they may in particular also contain additional water. This variant constitutes an nutritionally/physiologically valuable form of roughage.

In a particularly advantageous and preferred variant, the aleurone components and/or the aleurone are/is added in one of the above-mentioned forms to a conventional food, such as e.g., a food or combination of foods from the following food groups:

a) Basic foods such as flour or semolina made of wheat, rye, rice, corn or soya; fermented or non-fermented milk, animal or vegetable fats; fruit or fruit juice; vegetables or vegetable juice; pure water, mineral water or other enriched waters; meat; fish; etc.

b) Processed foods, such as cheese, quark or yoghurt; beverages, in particular milk drinks or juices; bakery products in particular bread or pastry; pasta; confectionery, in particular chocolate or jelly bears; breakfast cereals; sandwich spreads; bars; meat substitutes; sausage; etc.

c) Special foods with specific effects, such as functional food; pharma food; designer food; nutraceuticals; etc.

Pure aleurone, i.e. aleurone without a shell, is white and practically tasteless. In the aforementioned foods or other foods, this enables an addition of aleurone without altering either colour or taste, which leads, however, to an essential increase in the nutritional/physiological value of the food.

In another advantageous variant, the aleurone components and/or the aleurone in one of the aforementioned forms is added to a fodder, such as e.g., a fodder or combination of fodders from the following fodder groups:

a) Food for domestic animals or pets, such as dog food; cat food; rodent food; fish food; reptile food; amphibian food; bird food; etc.

b) Fodder for livestock, such as horse fodder; cattle fodder; pig fodder; sheep fodder; poultry fodder; etc.

In another special variant, the aleurone components and/or the aleurone in one of the aforementioned forms is added in a carrier which is neutral to the metabolism and in particular indigestible. This enables a dilution of the effective substances of the aleurone and a dosage in the form of capsules, bars, etc., when the indigestible carrier comprises an agent or a combination of agents from the following group; insoluble salts; waxes; starch; gelatine; chewing-gum; etc.

The aleurone components and/or the aleurone can also be added to a tasty carrier, in particular a luxury food, such as e.g., an agent or combination of agents from the following group: dextrose; maltose; fruit gum; etc.

In a process according to the invention for the manufacture of a variant of aleurone components and/or aleurone of the aforementioned kind, the aleurone components and/or the aleurone are/is added to a food or fodder in a liquid or powder form easily accessible to the human or animal organism, when the aleurone components and/or the aleurone are/is added to a processed food or a processed fodder during processing in a liquid or powder form easily accessible to the human or animal organism. Therefore, for an intentional weakening of the aleurone cells, a suitable procedural step of the processing of the conventional food can be used, when a separate step for the weakening of the aleurone cells prior to their being mixed in with the conventional food can thus be dispensed with.

A particular point in this context is that intact aleurone or intact aleurone cells or aleurone cell groups are mixed into a processed food or a processed fodder during a step in processing, when the food or fodder is exposed to at least one of the following influences or combinations thereof enzymatic influence; mechanical influence; in particular shearing, compression, expansion, etc.; thermal influence, in particular through steam and/or microwaves; chemical influence.

In a special variant of the process according to the invention, the mixing in of the total quantity of aleurone components and/or aleurone to be added is distributed among several procedural steps of the food or fodder processing. Thus, some of the aleurone cells added at an earlier stage can be weakened to a more severe extent than some of the aleurone cells added at a later stage. Accordingly, any combination of weakened/unweakened aleurone cells can be added to a food product and the food product can be subjected to any type of weakening treatment to weaken and/or further weaken the aleurone cells.

Preferably, the processing of the food or fodder takes place constantly, and the aleurone components and/or the aleurone are/is added constantly in at least one of the following forms during the processing of the food or fodder:

aleurone particle powder comprising aleurone cells and/or aleurone cell groups

aleurone juice from aleurone cell plasma

suspended aleurone cells and/or aleurone cell groups

diluted aleurone cell plasma

thickened aleurone cell plasma

granules, pellets, powder, etc. made from aleurone cell plasma.

The aforementioned variants according to the invention can be used as food supplements or fodder supplements.

Further advantages, features and means of using the invention are apparent from the following description of some examples, which are to be interpreted as non-restrictive.

Preparation of Wheat Aleurone

The raw material for the preparation of aleurone is a food-grade wheat bran from domestic wheat (Switzerland) containing 15% imported wheat. The aleurone was separated from the pericarp layer by means of a special mill and then isolated by several purification steps. Some samples were further reduced by micro milling on a friction roller mill (Buhler DNWA).

The aleurone preparations are made-up of clusters of 5 to 40 cells attached to the hyaline layer as well as minor amounts of pieces of pericarp layer and starch granules as major non-aleurone material (FIG. 2). The aleurone preparation with a lower purity is called Aleurone Standard Product-1 (ASP-1) and the more purified version Aleurone Standard Product-2 (ASP-2)

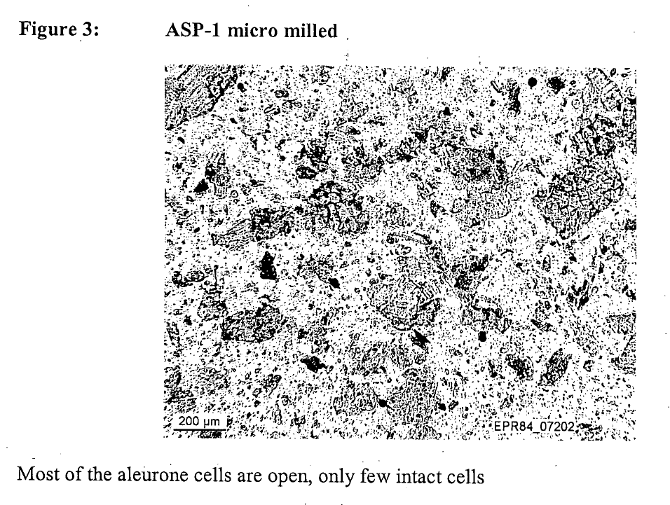

The micro milled (MM) preparations ASP-1 MM and ASP-2 MM are small flakes. The photomicrograph (FIG. 3) shows that about 70%-80% of the cells are ruptured and the original cell clusters appear as smears among some clusters of the original shape and structure.

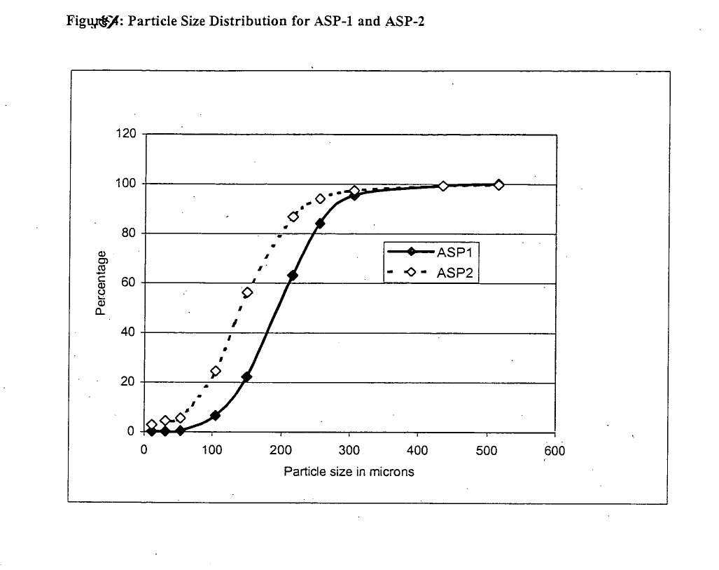

The aleurone preparations are free flowing powders with a color varying from light brown for ASP-1 to light yellow for the purer ASP-2. The micro milled samples are brighter than the standard preparations. The particle sizes vary between 100 and 250 μg (FIG. 4), the average particle size (X₅₀) is around 195μ for ASP-1 and 14μ for ASP-2.

In Vitro Digestion and Fermentation of Wheat Aleurone

To study the behavior of aleurone in the gastrointestinal tract, the digestibility and fermentability were analyzed by using a well established in vitro system by simulating digestion and fermentation in humans.

The in “In Vitro digestion model simulated the events taking place in the mouth, stomach, and small intestine. The digestion rates were 32% and 28% for ASP-1 and ASP-2, respectively, whereas only 13% of wheat bran has been digested.

High Fermentation Rate for Wheat Aleurone

The digested aleurone samples were slowly but almost completely degraded during “in vitro” fermentation. After 8 hours of fermentation, 80% of aleurone samples were degraded, whereas more than 50% of wheat bran was still unfermented after 24 hours. Only some small cell fragments of aleurone cells were observed under the microscope, indicating a high degree of degradation of these aleurone preparations by microorganism.

The main products of fermentation are short-chain fatty acids. ASP-2 had a higher production rate than wheat bran; the ratio of butyrate/propionate/acetate was 21:21:54 for both samples.

In accordance with the present invention, an easily accessible form of aleurone includes that compared to regular/untreated bran, the aleurone of the present invention has better bio availability (a) during digestion by human digestive enzymes; and (b) during fermentation by colonic bacteria.

The easy accessibility of the aleurone substances in treated aleurone is shown in FIGS. 2 and 3 where the aleurone separated from the bran and individual aleurone cells or aleurone cell clusters are more or less weakened/damaged as compared to the untreated bran containing the aleurone layer as shown in FIG. 1 in a single layer made up of the aleurone cells.

In relation to the different variants, “aleurone” is to be interpreted as a more or less disturbed (cell wall weakening) and, as the case may be, more or less complete mass (related to the natural composition of the aleurone) comprising aleurone cell groups and/or single aleurone cells and/or their components.

Use in Pasta

The aleurone is, for example, added in quantities of 8.5 to 50% as a “semolina substitute”. Through the use of aleurone in the form of primarily intact aleurone cell groups, a more or less pronounced sandiness can be achieved, depending upon the size of the cell groups, which in most cases is, however, more undesired than otherwise. Therefore, through the addition of aleurone, not only is it possible to improve the pasta from nutritional/physiological aspects, but also influence its sensory properties. Upon a use of pure, practically white aleurone, the appearance of the pasta remains largely unchanged. It has, moreover, transpired that upon the addition of aleurone in the form of larger cell groups (up to 100 aleurone cells or even more), the size of these cell groups decreases during the production or processing of the pasta. It can be proven that a “disagglomeration” of these cell groups takes place, and with a sufficiently intensive influence an at least partial weakening of the cell walls. As a result, any undesired sandiness is prevented. The pasta according to the invention thus corresponds to the eating habits among the population (one “cannot taste” the goodness).

Use in Cereals

In this context, the aleurone can be e.g., added in the form of flakes or soft and hard cereal bars, when e.g., a simple mixing in or coating aleurone constitutes a means of application.

Use in Liquid and Semi-Liquid Products

The aleurone can e.g., be added to yoghurt, sour milk, cream cheese or powder-based beverages (nectars). Here again, and depending upon the size of the cell groups, a more or less pronounced sandiness of the products is achieved. Moreover, the deposit reaction of the added aleurone is strongly dependent upon the size of the aleurone particles (cell components, individual components, cell groups).

Use in Beverage Powders

The aleurone can e.g., be added to breakfast drinks, energy drinks, athletes' drinks. Again, the accessibility of the aleurone components to the organism, on the one hand, and the sensory properties of the beverage, on the other, can be influenced by the state of the aleurone (cell components, individual cells, cell groups). For example, aleurone juice can be integrated by means of spraying into the finished beverage powder.

Use in Paste Products

The aleurone can also e.g., be mixed into sandwich spreads such as margarine, butter, sauces, dips, etc. Here again, the accessibility of the components of the aleurone to the organism, on the one hand, and the sensory properties of the beverage, on the other, can be influenced by the state of the aleurone (cell components, individual cells, cell groups).

Use in Bread and Bakery Products

In addition to the nutritional/physiological effect, and depending upon the quantity used (e.g., 8.5 to 20%) and state (cell components, individual cells, cell groups), the added aleurone can influence the developments of the dough, its rheology, baking reactions and baking results (properties of crumbs, sensory properties in the interior of the baked product).

Use in Boiling Extrusion and Cold Extrusion

The aleurone can be added in the extruder during a suitable procedural step of the processing of the product. Depending upon the state of the added aleurone (cell components, individual cells, cell groups), and according to the procedural step, the added aleurone can be processed to a more or less intensive extent, and thus changed. The added aleurone can therefore be exposed to a made-to-measure combination of enzymatic influence, mechanical influence, in particular shearing, compression, expansion, etc., or thermal influence, in particular through steam. Boiling or cold extrusion is also suitable for the manufacture of “pure aleurone products” in conjunction with a carrier such as starch, further grain bases (e.g., oat bran, flax, etc.) as separately administrable variants of food or fodder supplements in pellet or pressling form.

### SUMMARY

Through a use of “pure aleurone” in its various forms (cell components, i.e. aleurone mush, aleurone pulp, intact individual cells, cell groups), it is possible to influence the nutritional/physiological and sensory properties of the aleurone. A further influencing of these properties can be achieved through a deliberate omission of specific components (e.g., cell walls in destroyed aleurone, phytin, . . . ) of the aleurone. In this manner, it is possible to manufacture very specific foods and fodders with made-to-measure properties. Finally, additional non-aleurone components may be mixed in with the aleurone. This is of particular interest in the production of aleurone-based food supplements.

Finally, it should be mentioned by way of example that e.g., cellulose can also be added to the aleurone or aleurone-based product according to the invention. Whereas, however, cellulose has a practically pure roughage effect (hardly decomposable by intestinal bacteria), the addition of aleurone cell walls (unimpaired, weakened or destroyed) has a supplementary prebiotic effect. The aleurone cell walls predominantly comprising arabinoxylane function as a “breeding ground” or “food” e.g., for Bilfidus bacteria, whose metabolic products, in particular, butyric acid, assist in maintaining a healthy intestine.

We claim:

1. A composition comprising at least one aleurone component and aleurone extracted from bran, effective substances contained in the aleurone and their favourable physiological properties are suppliable to a human or animal in an easily accessible form.

2. The composition according to claim 1, wherein the bran is wheat bran.

3. The composition according to claim 1, wherein different aleurone components are contained in relative proportions which correspond to their natural relative proportions in the bran, wherein the bran is wheat bran.

4. The composition according to claim 1, wherein different aleurone components are contained in relative proportions which deviate from their natural relative proportions in the bran, wherein the bran is wheat bran.

5. The composition according to claim 1, wherein the aleurone component is in a form of aleurone cell groups.

6. The composition according to claim 5, wherein the aleurone cell groups comprise an average of up to about 100 aleurone cells.

7. The composition according to claim 6, wherein the aleurone cell groups comprise an average of up to about 5 to about 50 aleurone cells.

8. The composition according to claim 7, wherein the aleurone cell groups comprise an average of up to about 10 to about 20 aleurone cells.

9. The composition according to claim 5, wherein the aleurone cells of the aleurone cell groups have weakened cell walls.

10. The composition according to claim 9, wherein the walls of the aleurone cells have been weakened through enzymatic influence.

11. The composition according to claim 9, wherein the walls of the aleurone cells have been weakened through mechanical influence.

12. The composition according to claim 9, wherein the walls of the aleurone cells have been weakened through chemical influence.

13. The composition according to claim 9, wherein the walls of the aleurone cells have been weakened through thermal influence, wherein the thermal influence is at least one of steam treatment and microwave treatment.

14. The composition according to claim 9, wherein the walls of the aleurone cells have been weakened through a combination of enzymatic, mechanical, chemical or thermal influence.

15. The composition according to claim 1, wherein the aleurone components are in the form of destroyed aleurone cells, and comprise both the total cell content and the entire destroyed cell walls of the aleurone.

16. The composition according to claim 4, wherein the aleurone component is in the form of destroyed aleurone cell, and comprises at least one part of the entire cell content and/or at least one part of the destroyed cell wall of the aleurone.

17. The composition according to claim 1, wherein no other components are present with the exception of at least one aleurone component.

18. The composition according to claim 17, wherein at least one of the aleurone component and aleurone comprises aleurone cell groups in flake, granule, pressling or powder form.

19. The composition according to claim 18, wherein at least one of the aleurone component and aleurone contains at least one destroyed aleurone cell.

20. The composition according to claim 17, wherein at least one of the aleurone component and aleurone comprises only cell content which originates from aleurone cells in liquid form.

21. The composition according to claim 18, wherein the cell content is thickened to make a thick fluid.

22. The composition according to claim 20, wherein the cell content is thickened to make a diluted to make a runny fluid.

23. The composition according to claim 17, wherein at least one of the aleurone component and aleurone comprises only the cell content originates from which originates from aleurone cell group in its dried form, wherein the dried form is in flake, granule, pressling, or powder form.

24. The composition according to claim 17, wherein at least one of the aleurone component and aleurone comprises only destroyed cell walls originating from dried form in flake, granule, pressling or powder form.

25. The composition according to claim 17, wherein at least one of the aleurone component and aleurone comprises only destroyed cell walls originating from aleurone cells and water.

26. The composition according to claim 1 further comprises a food component.

27. The composition according to claim 26, wherein the food component is at least one of a) basic food which is at least one of flour or semolina made from wheat, rye, rice, corn or soya; fermented or non-fermented milk; animal or vegetable fats; fruit or fruit juice; vegetable or vegetable juice; pure water, mineral water or other enriched water; meat; and fish; b) processed food which is at least one of cheese, quark or yoghurt; beverages, milk, or juices; bakery products, bread or pastry; noodles or pasta; confectionery, chocolate, or jelly; breakfast cereals or bars; sandwich spreads; meat substitute; and sausage; and c) special food with specific effects which is at least one of functional food; pharma food; designer food; and nutraceuticals.

28. The composition according to claim 1 further comprises a fodder component.

29. The composition according to claim 28, wherein the fodder component is at least one of a) food for domestic animals or pets which is at least one of dog food; cat food; rodent food; fish food; reptile food; amphibian food; bird food; and b) fodder for livestock which is at least one of horse fodder, cattle fodder, pig fodder, sheep fodder, and poultry fodder.

30. The composition according to claim 1 further comprises a carrier which is neutral to the metabolism.

31. The composition according to claim 30, wherein the carrier is an indigestible carrier.

32. The composition according to claim 30, wherein the indigestible carrier is an agent or a combination of agents at least one of insoluble salts, waxes, starch, gelatine, and chewing-gum.

33. The composition according to claim 1, further comprises a tasty carrier.

34. The composition according to claim 33, wherein the tasty carrier comprises at least one agent which is at least one of dextrose, maltose, and fruit gum.

35. A method for manufacturing the composition of claim 1 comprising adding the aleurone component and/or the aleurone to a food component or fodder component in a liquid or powder form which is easily accessible to the human or animal organism.

36. A method for manufacturing the composition of claim 1 comprising adding at least one aleurone component and aleurone to a processed food or processed fodder during its processing, in a liquid or powder form which is easily accessible to the human or animal organism.

37. A method for manufacturing the composition of claim 1, comprising: adding intact aleurone or intact aleurone cells or aleurone cell groups to a processed food or a processed fodder during a procedural step of processing, when the food or fodder is exposed to at least one of enzymatic influence, mechanical influence, thermal influence, and chemical influence, wherein the mechanical influence is shearing, compression or expansion, and wherein the thermal influence is at least one of steam and microwave.

38. The method according to claim 36, wherein the entire quantity of the aleurone component and/or the aleurone is added to the food component or fodder component in more than one step.

39. The method according to claim 36, wherein the aleurone component and/or the aleurone is constantly added to the food component or the fodder component in at least one of the following forms during the processing a) aleurone particle powder comprising aleurone cells and/or aleurone cell groups; b) aleurone juice comprising aleurone cell plasma; c) suspended aleurone cells and/or aleurone cell groups; d) diluted aleurone cell plasma; e) thickened aleurone cell plasma; f) granules, pellets, or powder comprising dried aleurone cell plasma.

40. A method of administering to a human or animal comprising administering a food supplement or fodder supplement containing the composition of claim 17.

41. The method according to claim 35, wherein after the addition of the aleurone component to the food component, the food is subjected to treatment to weakening or further weakening of the aleurone component.

42. A method for manufacturing a composition which comprises adding at least one of weakened aleurone cells and unweakened aleurone cells to a food product, and subjecting the food product to at least one type of weakening treatment to weaken or further weaken the aleurone cells.

43. A method of administering a food product to a human or animal comprising administering to a human or animal the composition manufactured by the method of claim 42.
